(19) United States
(12) Patent Application Publication
(10) Pub. No.: US 2009/0155183 A1
(43) Pub. Date: Jun. 18, 2009

(54) Sensors for the detection of diols and carbohydrates

(75) Inventor: Heather A. Clark, Lexington, MA (US)

Correspondence Address: ROPES & GRAY LLP, PATENT DOCKETING 39/41, ONE INTERNATIONAL PLACE, BOSTON, MA, 02110-2624, US

(73) Assignee: The Charles Stark Draper Laboratory, Inc., Cambridge, MA (US)

(21) Appl. No.: 12/156,959

(22) Filed: Jun. 6, 2008

Related U.S. Application Data
(60) Provisional application No. 60/933,724, filed on Jun. 8, 2007.
(60) Provisional application No. 61/130,195, filed on May 29, 2008.

(51) Int. Cl. A61K 49/00; G01N 21/00; C12Q 1/02; C12Q 1/54

(52) U.S. Cl. 424/9.6; 436/164; 435/29; 435/14; 977/774

(57) ABSTRACT

The systems and methods disclosed herein include a sensor particle for detecting the presence of a chelatable analyte, such as glucose, wherein the sensor comprises a chromophore and a fluorescent component, such as a quantum dot. The sensor particle further comprises moieties that bind both a clelatable analyte and chromophore reversibly and competitively. In the presence of the chelatable analyte, the moieties bind the analyte, and release the chromophore. The chromophore absorbs photons of one wavelength in a free state but of a different wavelength in a bound state, and is selected to operate with the fluorescent component such that the chromophore absorbs emissions of the fluorescent substance in only one of the bound and unbound states. In certain aspects, the invention comprises methods for detecting the presence of a chelatable analyte in a medium such as water, blood plasma and urine, using the sensor particles of the invention.

### CROSS REFERENCE TO RELATED APPLICATIONS

This application claims the benefit of U.S. Provisional Patent Application Ser. No. 60/933,724 filed on Jun. 8, 2007 and the U.S. Provisional Patent Application entitled Sensors for the Detection of Diols and Carbohydrates by inventor Heather Clark, filed on May 29, 2008. The teachings of all of the referenced applications are incorporated by reference in their entirety.

### BACKGROUND OF THE INVENTION

Diabetes has become a national health-care crisis. According to the 2005 National Diabetes Fact Sheet, an estimated 20.8 million people in the United States suffer from diabetes. The costs associated with diabetic care are also astronomical, with an estimated $132 billion dollars spent in 2002. As a result of a seminal study highlighting the benefits of tight glycemic control, the American Diabetes Association recommends that patients with diabetes should try to control their glucose levels to be as close to normal as possible. With tight glycemic control, the complications associated with diabetes, such as heart disease, blindness and amputation are significantly reduced. Self-monitoring of glucose is essential for regulation, particularly for those with Type 1 diabetes. It is often performed through a finger-stick method three times or more per day. The need to draw blood, even in small quantities, multiple times a day is not desirable.

A continuous monitoring system would be highly advantageous for patients and healthcare providers alike. It has become the goal of glucose sensor research, and continuous monitoring systems of many varieties are pursued by countless researchers in the field. The benefits of continuous monitoring over the finger-stick method are numerous. First, the finger-stick method is both painful and inconvenient for the patient, which can lead to noncompliance. Second, a single-point measurement gives static information on the concentration of blood glucose, with no knowledge of the trend, or in other words, whether the level is going up or down. Third, monitoring at night, a time when levels could dip dangerously low, is either not performed or especially inconvenient. Continuous monitoring systems have been pursued in many different forms, and some are commercially available, such as the Guardian RT from Medtronic MiniMed (Northridge, Calif.), and the Gluco Watch Biographer from Animas (West Chester, Pa.). Both of these systems work by sampling glucose from the interstitial space, the extracellular space in the dermis, rather than the blood. Currently, they are approved as monitors to track trends in glucose but highs and lows are verified by a finger-stick test. Some reports have shed doubt on the accuracy of nighttime monitoring in patients whose glucose is tightly controlled.

Commercially available systems for continuous or finger-stick measurements rely on electrochemical biosensors. Glucose oxidase is the most well-known of the biological recognition units, and the enzyme provides a highly selective sensor platform. Enzyme-based sensors are difficult to implement as implantable glucose sensors, since the enzyme limits itself in a confined environment. Oxygen, required for function, regionally depletes, and hydrogen peroxide, a by-product of the reaction, can lead to enzyme degradation. Most often the read-out is electrode-based, which is an added challenge for miniaturization and biological implantation. Nano- and microscale optical sensors have also been demonstrated, but typically lack the selectivity and robustness to replace traditional techniques.

There is still a need for a continuous, non-invasive method for glucose monitoring, especially one that is easy to use, highly accurate and pain-free.

### SUMMARY OF THE INVENTION

This invention discloses a sensor particle for detecting the presence of a chelatable analyte, such as glucose, comprising a quantum dot, a polymer matrix comprising a polymer including moieties that bind the chelatable analyte and a chromophore associated with the polymer matrix that binds to the moieties in the absence of the chelatable analyte. In some embodiments, photons emitted by the quantum dot in an excited state are absorbed by the chromophore in an unbound state but not by the chromphore in a bound state. The moieties may bind the chelatable analyte and chromophore reversibly and competitively. In certain embodiments, the moieties are boronic acids or boronic esters. In some embodiments, one or more components of the sensor, such as the moieties and/or chromophore, are covalently bound to or associated with the polymer matrix. In some embodiments, the sensor particles further comprise a biocompatible layer.

In certain aspects, the invention comprises methods for detecting the presence of a chelatable analyte in a medium using the sensor particles of the invention. In certain embodiments, the chelatable analyte is glucose and the medium is selected from water, blood, plasma and urine. In certain embodiments, the invention comprises a method for detecting the presence of a chelatable analyte in an animal. In certain such embodiments, the sensor particle is implanted in the dermis or epidermis and the chelatable analyte, such as glucose, is monitored.

### BRIEF DESCRIPTION OF THE DRAWINGS

FIG. 1. Sensor particle 3 with a. chromophore 2 bound to moiety 1, wherein the bound chromophore emits photons 4 at one wavelength and b. moiety 1 bound to analyte 5 wherein the unbound chromophore 2 emits photons at a second wavelength 6.

FIG. 2. Sensor particle 3 with a. chromophore 2 bound to moiety 1, wherein the bound chromophore 2 does not absorb photons 4 emitted by the quantum dot and/or fluorescent dye 7 and b. moiety 1 bound to analyte 5 wherein unbound chromophore 2 absorbs photons 4 emitted by quantum dot and/or fluorescent dye 7.

FIG. 3. Sensor particle 3 with a. chromophore 2 bound to moiety 1, wherein the bound chromophore absorbs photons 4 emitted by the quantum dot and/or fluorescent dye 7 and b. moiety 1 bound to analyte 5 wherein unbound choromophore 2 absorbs photons 4 emitted by quantum dot and/or fluorescent dye 7.

FIG. 4. An exemplary embodiment of the competitive interaction of a boronic acid (moiety which binds the chelatable analyte ) with alizarin (chromophore), or glucose (analyte).

FIG. 5. Spectral signature of the components of a GSQD; a. overlap of normalized alizarin absorbance and quantum dot emission, b. individual contribution of the two components of the inner filter effect at high and low glucose concentration and the resulting overall fluorescence signal.

FIG. 6. Wide field fluorescence microscopic image of a suspension of sensor particles.

FIG. 7. Nanometer-sized sensor particles demonstrating the inner filter effect wherein a. the absorbance changes from purple to yellow depending on the binding state of the chromophore, b. the same samples under UV excitation wherein the sample that was visually purple does not absorb the 525 nm emission of the quantum dots and fluoresces brightly, while the yellow sample absorbs the fluorescence emission of the quantum dot and has minimal emission.

FIG. 8. Evaluating response to glucose, the sensor particles containing the essential sensing components, alizarin, pyrene boronic acid and additive, was immobilized to the bottom of a micro-well for calibration. Response to glucose and fructose was measured, the average±SEM is shown, where n=6 and n=8 for control and monosaccharides, respectively.

FIG. 9. Measuring the degree of cytotoxicity of sensor particles by incubating the particles overnight with HEK 293 calls and measuring the degree of cellular injury with an MTT assay. Results of particle sensors are compared to other particles, e.g., gold, latex.

### DETAILED DESCRIPTION

Disclosed are sensor particles for the detection of chelatable analytes, e.g., glucose. The sensor particles comprise a polymer matrix, moieties which bind a chelatable analyte, and a component that emits or absorbs photons of a particular wavelength either in the presence of absence of the chelatable analyte. In an exemplary embodiment, a chromophore absorbs photons of one wavelength when bound to the moieties of the sensor and another wavelength when unbound from the moieties. When the chromophore-bound moieties are exposed to the chelatable analyte, the chromophore is released and the chelatable analyte binds to the moieties. The free chromophore appears as a different color than the bound chromophore, a change which can be monitored visually or with spectrophotometric instrumentation. In an alternate exemplary embodiment, wherein the inner-filter effect is employed, the sensor particle of the preceding embodiment further comprises a fluorescent dye and/or quantum dot. The fluorescent dye and/or quantum dot absorbs a broad range of wavelengths and emits photons of a narrow range of wavelengths. The fluorescence emitted by the fluorescent component is either absorbed or not absorbed depending on the presence of the chelatable analyte. For example, when the chelatable analyte is bound to the moieties of the sensor, the fluorescence of the quantum dot is absorbed while no absorbance occurs in the absence of the chelatable analyte.

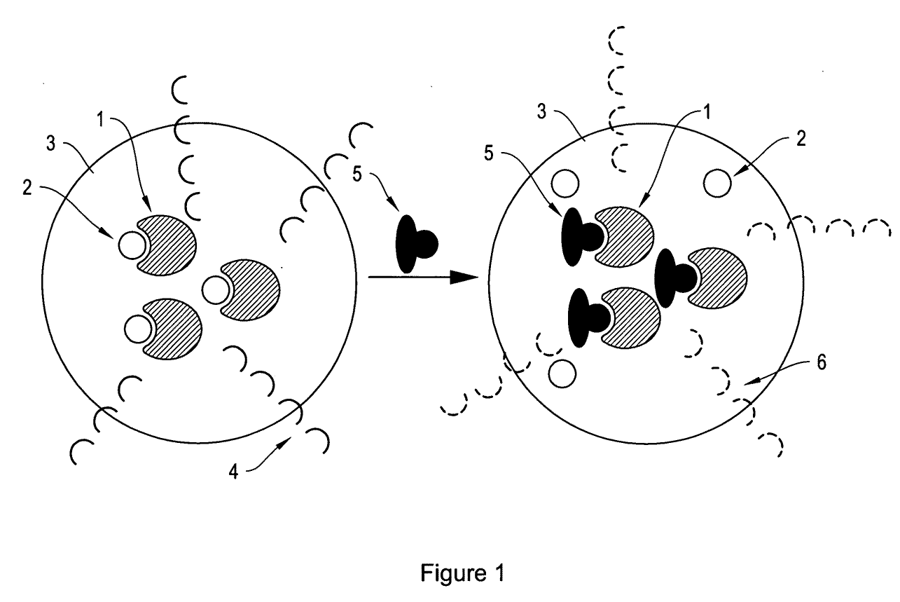

In certain embodiments, the sensor particle for detecting the presence of chelatable analytes comprises a polymer matrix comprising a polymer including moieties that bind the chelatable analyte and a chromophore associated with the polymer matrix that binds to the moieties in the absence of the chelatable analyte. In certain embodiments, the chelatable anaylte is glucose and the moieties bind glucose and the chromophore reversibly and competitively. In an exemplary embodiment, the sensor particle 3 comprises a polymer matrix with moieties 1 that can bind both a chromophore 2 and glucose 5 (FIG. 1). In a first mode, the moieties 1 are bound to a chromophore 2 and the chromophore, in its bound mode, absorbs photons at a first wavelength 4. In a second mode, when the sensor particle 3 is contacted with glucose 5, the glucose 5 binds to the moieties 1, displacing the chromophore 2 which, in its unbound state, absorbs photons at a second wavelength 6. In certain embodiments, the sensor 3 is monitored visually to determine a change in the color of the chromophore 2. In certain embodiments, the sensor 3 is monitored with spectrophotometric instrumentation to determine the emission spectra of the chromophore 2.

In certain embodiments, the sensor particle for detecting the presence of a chelatable analyte comprises a fluorescent component, a polymer matrix comprising a polymer including moieties that bind the chelatable analyte and a chromophore associated with the polymer matrix that binds to the moieties in the absence of the chelatable analyte. In certain embodiments, the sensor particle emits photons with an inner filter effect. The inner-filter effect has been documented as a way to increase the signal intensity and concomitant sensitivity of ion-selective optical sensors (optode). In brief, a secondary, inert fluorescent component is added to the polymer matrix of the optode. When the concentration of analyte in the optode changes, the fluorescence intensity of the inert dye itself does not respond, however the absorbance of the sensor does. Because the fluorescence emission has been carefully chosen to overlap with the absorbance spectrum of the sensor, the emission from the inert dye is then absorbed by the sensor. The attenuation of the fluorescence output of the inert dye is therefore directly related to the concentration of the ion of interest in solution.

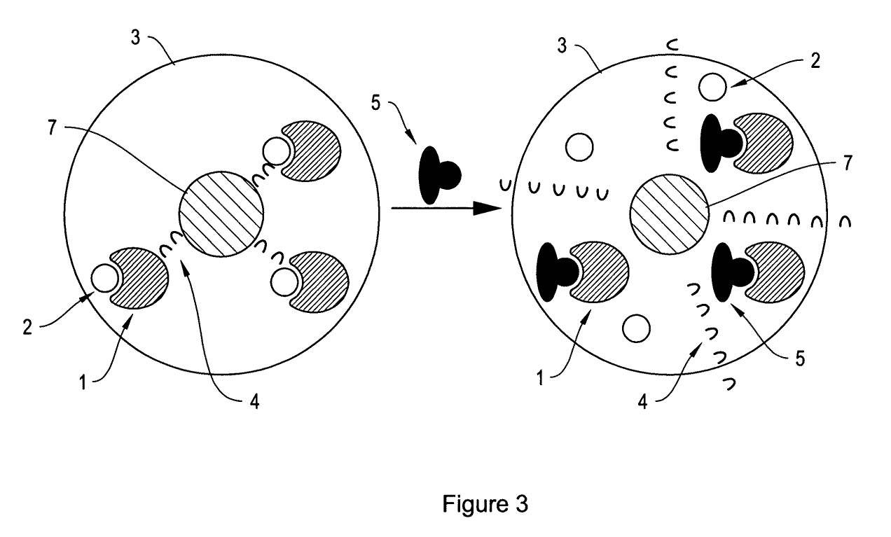

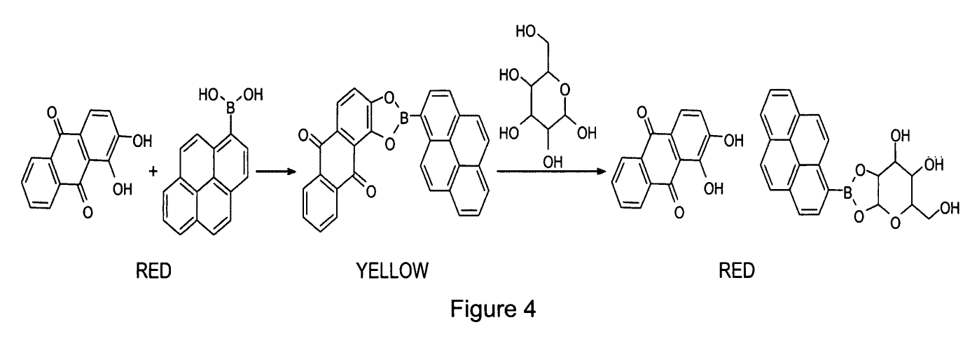

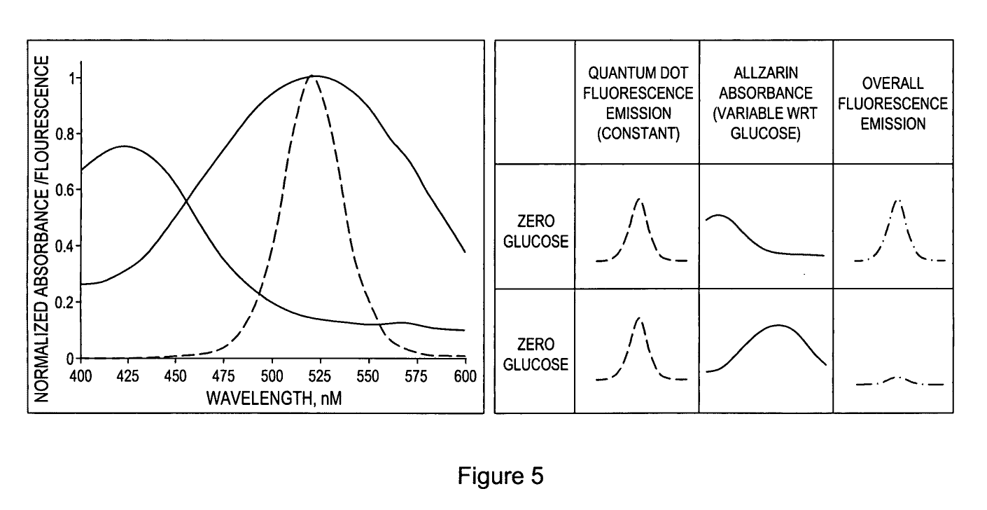

In certain embodiments, the chelatable analyte is glucose and the moieties bind glucose and the chromophore reversibly and competitively. In certain embodiments, the fluorescent component is selected from one or more quantum dots and/or fluorescent dyes 7. In certain such embodiments, a sensor particle 3 comprises a fluorescent component 7, and a polymer matrix with moieties 1 that can bind both a chromophore 2 and glucose 5. In certain such embodiments, the fluorescent component 7 absorbs a broad range of wavelengths of photons but emits a narrow range of wavelengths of photons. The fluorescent component 7 is activated by exciting with a light source, e.g., UV light. The fluorescence emitted from the excited fluorescent component 7 is either absorbed by a component of the sensor, e.g., the chromophore 2 or the glucose-moiety complex, or emitted from the sensor 3 without being attenuated. In certain embodiments, photons 4 of the fluorescent component 7 are absorbed when the chromophore 2 is bound to the moieties 1 (FIG. 3, left). In certain such embodiments, the absence of fluorescence emitted from the sensor particle 3 indicates an absence of glucose molecules 5, i.e. glucose molecules are not bound to the moieties of the sensor. In such embodiments, when glucose 5 is introduced, the moieties 1 bind glucose 5, releasing the chromophore 2. The photons 4 of the fluorescent component 7 are no longer absorbed by a component of the sensor, FIG. 3, right. By detecting the emitted photons, the amount of bound glucose can be calculated relative to a standard.

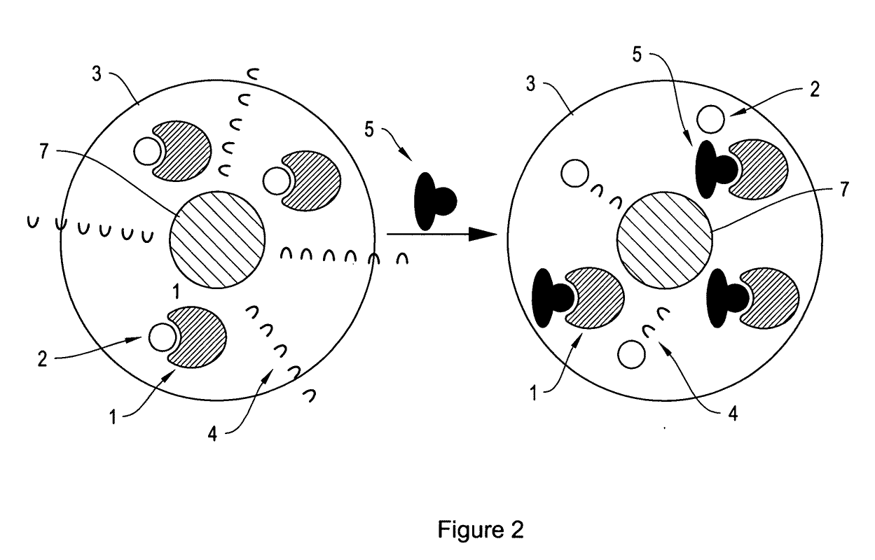

In certain embodiments, a component of the sensor, e.g., the chromophore 2 or the glucose-moiety complex, absorbs photons 4 of the fluorescent component 7 when unbound from the moieties 2 (FIG. 2, right). In certain such embodiments, the detection of photons 4 from the sensor 3 indicates the absence of glucose 5, i.e. glucose molecules are not bound to the moieties of the sensor. In certain such embodiments, when the sensor 3 is contacted with glucose 5, the moieties 1 release the chromophore 2 and bind glucose 5. In such embodiments, the photons 4 of the fluorescent component 7 are not absorbed when glucose 5 is bound to the moieties 1 such that the detection of photons 4 emitted from the sensor particle 3 indicates the presence of glucose 5.

In certain embodiments, the sensors of the present invention may be used to detect and measure the presence of a wide variety of chelatable analytes, e.g., sugars and related compounds, in a solution, in vitro or in vivo. The sensor may be located within a cell, i.e., intracellular, or exterior to a cell, i.e., extracellular. In certain embodiments, the sensor is in contact with the cell membrane such as within a cell or exterior to a cell. Exemplary chelatable analytes for detection by the sensor of the present invention include sugars such as glucose, mannose, and other monosaccharides, sialic acid, lactic acids, aminosugars, such as glucosamine, disaccharides, trisaccharides, oligosaccharides, sugar-amino acids, sugar-peptides and glycoproteins. Other exemplary chelatable analytes include, but are not limited to, glycerol, dopamine, catechols, ascorbic acid, polyols, diols such as 1,4-anhydroerythritol and ethylene glycol. The concentration range of chelatable analytes which is typically of interest in biological samples is 0-25 mM, such as from 5-20 mM, such as from 5-10 mM, such as from 0-5 mM.

In certain embodiments, the moieties that bind the chelatable analytes comprise a dihydroxide component, e.g., boron and alkali earth dihydroxides. Complexation of sugars, for example, with boron and alkali earth dihydroxides has been reported in, among other sources, [S. A. Barker et al., Carbohydrate Research, 26 (1973) 33-40; N. Roy et al., Carbohydrates Research, 24 (1972) 180-183]. A variety of different boronic acids, having the structure RB(OH)₂ may be used to chelate the analyte. R can be, for example, an aryl or a saturated or unsaturated alkyl moiety, either of which can be substituted or unsubstituted and can contain one or more heteroatoms, e.g., N, S, O, P, B, F, Br. In certain embodiments, a boronic ester is used to chelate the analyte. Boronic esters have the molecular formula RB(OR′)₂ wherein R′ is typically an alkyl group and R can be defined as above. Under aqueous conditions, many boronic esters hydrolyze to form boronic acids. Therefore, OR′ groups that hydrolyze to OH are of use in the present invention. The two R′ groups of the ester may be linked to form a cyclic structure, e.g., —CH₂CH₂—. In certain embodiments, the moieties are selected from one ore more aromatic or aliphatic boronic esters. In certain aspects, boronic acids are appended with substituents that affect the pKa such as electron withdrawing groups or electron donating groups. In certain embodiments the pKₐ of the boronic acid will change the dynamic range of the sensor. In certain embodiments the dynamic range of the sensor relates to the affinity for an analyte, such as glucose. In certain embodiments, the moieties are selected from one or more aromatic or aliphatic boronic acids. Exemplary boronic acid moieties of the invention include phenyl boronic acid, butyl boronic acid, (3,5-dichlorophenyl)boronic acid, [3,5-bis(trifluoromethyl)phenyl]boronic acid, and (4-bromophenyl)boronic acid.

In certain embodiments, the moieties of the sensor which chelate the analytes comprise a metal ion. The ability of sugars, for example, and other molecules to form chelate complexes with metal ions in aqueous solution is well known (general review by: Whitfield, D. M. et al., “Metal coordination to carbohydrates. Structure and Function,” Coord. Chem. Reviews 122, 171-225 (1993) and Angya, S. J. Complexes of Metal Cations with Carbohydrates in Solution, in “Advances in Carbohydrate Chemistry and Biochemistry,” Academic Press, Inc. 1989, pp. 1-4). The complexation of Cu(II) with various sugar α-amino acids is described by M. Angeles Diaz-Diez et al., Transition Met. Chem. 20, 402-405, 1995. Sugar-α-amino acid compounds will also form complexes with Co(II), Ni(II), Zn(II) and Cd(II) (M. Angeles Diaz-Diez et al., J. Inorg. Biochem. 56, 243-247, 1994). Additionally, complexes of various sugars with vanadium, molybdenum, tungsten, aluminum, iron, barium, magnesium, and strontium are known (Sreedhara, A. et al., Carbohydrate Res. 264, 227-235, 1994; Caldeira, M. M. et al., Inorg. Chim. Acta. 221, 69-77, 1994; Tonkovic, M. and Bilinski, H., Polyhedron 14, 1025-1030, 1995; Nagy, L. et al., Inorg. Chim. Acta. 124, 55-59, 1986; Tajmir-Riahi, H. A., Inorg. Chim. Acta. 119, 227-232, 1986; and Tajmir-Riahi, H. A., J. Inorg. Biochem., 24, 127-136, 1985.

In certain embodiments, the moieties that bind the chelatable analytes are covalently conjugated to the polymer matrix. In certain embodiments, the moieties are covalently conjugated to the matrix, for example, through a linker molecule. In an exemplary embodiment, the moieties comprise aryl boronic acids which are covalently conjugated to the polymer matrix through ester linkages originating at an aryl atom or the aryl boronic acid. Other exemplary linkages include amides, ethers, sulfonates, thioethers, thioesters and carbonates. In certain embodiments, the moieties are covalently bound to the polymer matrix through a bond such as a single or double bond. In certain exemplary embodiments, the aryl boronic acids are covalently bound to the polymer matrix through a single bond originating from an aryl atom or the aryl boronic acid.

In certain embodiments, the chromophore of the sensor is any molecule that binds reversibly to the moieties of the sensor, e.g., the chromophore alizarin binds boronic acids, and absorbs photons of the fluorescent component in a first state and does not absorb photons of the fluorescent component in a second state. The states of the chromophore include bound to the moieties and unbound from the moieties. For example, the chromophore alizarin absorbs at a first wavelength when unbound and a second wavelength when bound to a boronic acid. In certain embodiments, the chromophore, e.g., alizarin, is selected from any dye that binds boronic acid moieties, preferably having absorbance/fluorescence properties that differ in the bound vs. the free state. When a suitable chelatable analyte is present, the boronic acid releases the chromophore and binds the analyte. Additional FDA approved dyes and colored drugs are described in the Code of Federal Regulations (CFR) for Food and Drugs (see Title 21 of CFR chapter 1, parts 1-99). A wide variety of chromophores and fluorescence sources may be used, e.g., paired so that the absorbance wavelength of the unbound chromophore substantially matches the wavelength of the fluorescent component's photon emissions, e.g., so as to absorb the emissions in an unbound state. The table below lists a number of suitable chromophores, their Chemical Abstract Service (CAS) Registration Numbers, colors and absorption maxima. In certain embodiments, the chromophore is derivatized in such a manner that it can bind with the chelating moiety of the sensor.

| Chromophore | CAS Reg. No. | Color | Abs. Max. |
|---|---|---|---|
| Yellow No. 5 | 1934-21-0 | yellow | 428 |
| β-carotene | 7235-40-7 | orange | 466 |
| Rifampin | 3292-46-1 | red | 475 |
| Yellow No. 6 | 2783-94-0 | yellow | 480 |
| Tetracycline | 60-54-8 | yellow | N/A |
| Red No. 40 | 25956-16-6 | red | 502 |
| Red No. 3 | 16423-68-0 | red | 524 |
| Blue No. 2 | 860-22-0 | blue | 610 |
| Evan's blue | 314-13-6 | blue | 610 |
| Green No. 3 | 2353-45-9 | green | 628 |
| Blue No. 1 | 2650-18-2 | blue | 630 |
| Methylene blue | 7220-79-3 | Blue | 668/609 |
| Indocyanine green | 3599-32-4 | Green | 800 (mostly IR) |

In certain embodiments, the chromophore is covalently conjugated to the polymer matrix and comprises a reactive site that binds reversibly with the chelatable analyte selective moieties. In an exemplary embodiment, the chromophore is alizarin, and the alizarin is covalently bound to the polymer matrix through a linker or bond. In certain embodiments, the linker is an ester amide, ether, sulfonate, thioether, carbonate or thioester originating from an aromatic carbon of the alizarin. In certain embodiments, the chromophore is covalently conjugated through a bond to the polymer matrix. In certain embodiments, the bond or linkage between the chromophore and the polymer matrix does not interfere with the ability of the chromophore to bind to the chelatable analyte. For example, in the case of alizarin, the linkage or bond to the polymer matrix originates from a ring of the polycyclic ring system that does not bear the hydroxy groups. In certain such embodiments, the hydroxyl groups of the alizarin are unimpeded from interacting with the chelatable analyte.

In certain embodiments, the polymer matrix of the sensor comprises poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, silicones, polyalkylenes such as polyethylene, polypropylene, and polytetrafluoroethylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) jointly referred to herein as “polyacrylic acids”), and copolymers and mixtures thereof, polydioxanone and its copolymers, polyhydroxyalkanoates, poly(propylene fumarate), polyoxymethylene, poloxamers, poly(ortho)esters, poly(butyric acid), poly(valeric acid), poly(lactide-co-caprolactone), trimethylene carbonate, polyvinylpyrrolidone, and the polymers described in Shieh et al., 1994, J. Biomed. Mater. Res., 28, 1465-1475, and in U.S. Pat. No. 4,757,128, Hubbell et al., U.S. Pat. Nos. 5,654,381; 5,627,233; 5,628,863; 5,567,440; and 5,567,435. Other suitable polymers include polyorthoesters (e.g. as disclosed in Heller et al., 2000, Eur. J. Pharm. Biopharm., 50:121-128), polyphosphazenes (e.g. as disclosed in Vandorpe et al., 1997, Biomaterials, 18:1147-1152), and polyphosphoesters (e.g. as disclosed in Encyclopedia of Controlled Drug Delivery, pp. 45-60, Ed. E. Mathiowitz, John Wiley & Sons, Inc. New York, 1999), as well as blends and/or block copolymers of two or more such polymers. The carboxyl termini of lactide- and glycolide-containing polymers may optionally be capped, e.g., by esterification, and the hydroxyl termini may optionally be capped, e.g., by etherification or esterification. In certain embodiments, the polymer comprises or consists essentially of polyvinyl chloride (PVC), polymethyl methacrylate (PMMA) or decyl methacrylate or copolymers or any combination thereof.

In certain embodiments, the polymer matrix of the sensor comprises a biocompatible layer, e.g., selected from poly(caprolactone) (PCL), ethylene vinyl acetate polymer (EVA), poly(ethylene glycol) (PEG), poly(vinyl acetate) (PVA), poly(lactic acid) (PLA), poly(glycolic acid) (PGA), poly(lactic-co-glycolic acid) (PLGA), polyalkyl cyanoacrylate, polyethylenimine, dioleyltrimethyammoniumpropane/dioleyl-sn-glycerolphosphoethanolamine, polysebacic anhydrides, polyurethane, nylons, or copolymers thereof. In certain embodiments, the biocompatible layer is disposed on the exterior of the sensor such as disposed around the polymer matrix and chromophore and optional component, such as a fluorescent dye and/or quantum dot. In polymers including lactic acid monomers, the lactic acid may be D-, L-, or any mixture of D- and L-isomers. In certain aspects, the biocompatible layer of the sensor particle comprises a PEG-lipid. In certain embodiments, the lipid tail self-inserts into the lipophilic polymer matrix during fabrication, leaving the PEG headgroup on the surface of the sensor, e.g., to provide a hydrophilic, biocompatible coating that can be penetrated by the analyte. In certain embodiments, different chemical moieties, such as amines, can be put on the surface or further modified to attach antibodies or other recognition units.

The terms “biocompatible polymer,” “biocompatible layer” and “biocompatibility” when used in relation to polymers are art-recognized. For example, biocompatible polymers include polymers that are neither themselves toxic to the host (e.g., a cell or an animal such as a human), nor degrade (if the polymer degrades) at a rate that produces monomeric or oligomeric subunits or other byproducts at toxic concentrations in the host. Consequently, in certain embodiments, toxicology of a biodegradable polymer intended for intracellular and/or in vivo use, such as implantation or injection into a patient, may be determined after one or more toxicity analyses. It is not necessary that any subject composition have a purity of 100% to be deemed biocompatible. Hence, a subject composition or layer may comprise 99%, 98%, 97%, 96%, 95%, 90% 85%, 80%, 75% or even less of biocompatible polymers, e.g., including polymers and other materials and excipients described herein, and still be biocompatible.

The polymer matrix of the sensor may comprise a plasticizer, such as dioctyl sebacate (DOS), o-nitrophenyl-octylether, dimethyl phthalate, dioctylphenyl-phosphonate, dibutyl phthalate, hexamethylphosphoramide, dibutyl adipate, dioctyl phthalate, diundecyl phthalate, dioctyl adipate, dioctyl sebacate, or other suitable plasticizers. In certain embodiments, the plasticizer is poly(glycerol sebacate), PGS.

In certain embodiments, e.g., particularly where the polymer is biocompatible, a biocompatible plasticizer is used. The term “biocompatible plasticizer” is art-recognized, and includes materials which are soluble or dispersible in the relevant polymer, which increase the flexibility of the polymer matrix, and which, in the amounts employed, are biocompatible. Suitable plasticizers are well known in the art and include those disclosed in U.S. Pat. Nos. 2,784,127 and 4,444,933. Specific plasticizers include, by way of example, acetyl tri-n-butyl citrate (c. 20 weight percent or less), acetyltrihexyl citrate (c. 20 weight percent or less), butyl benzyl phthalate, dibutylphthalate, dioctylphthalate, n-butyryl tri-n-hexyl citrate, diethylene glycol dibenzoate (c. 20 weight percent or less) and the like.

In certain embodiments, the sensor particle for detecting the presence of glucose comprises: a quantum dot, a polymer matrix comprising a polymer appended with moieties that selectively bind glucose, a chromophore associated with the polymer matrix that binds the moieties in the absence of glucose and a biocompatible layer.

In certain embodiments, additives to the polymer matrix make the extraction of the analyte (e.g., glucose) into the polymeric matrix more efficient. In certain embodiments, the addition of amine-based additives to the matrix lowers the effective dynamic range of the sensor particles. In certain embodiments, the addition of amines to the polymer matrix increases the affinity of the polymer matrix for the analyte, e.g., glucose.

In certain embodiments, the sensor comprises one or more quantum dots. Quantum dots are fluorescent semiconductor nanocrystals having a characteristic spectral emission, which is tunable to a desired energy by selection of the particle size, size distribution and composition of the semiconductor nanocrystal. The quantum yield of quantum dots is high, with reports of greater than 90% efficiency in cladded quantum dots, photobleaching is minimal, and a single quantum dot can be continuously tracked for minutes to hours. There is a wide range of colors available, all with the same excitation wavelengths, and very narrow emission bandwidths. The emission spectra of a population of quantum dots have linewidths as narrow as 25-30 nm, depending on the size distribution heterogeneity of the sample population, and lineshapes that are symmetric, gaussian or nearly gaussian with an absence of a tailing region. Advantageously, the range of excitation wavelengths of the quantum dots is broad. Consequently, this allows the simultaneous excitation of varying populations of quantum dots in a system having distinct emission spectra with a single light source, e.g., in the ultraviolet or blue region of the spectrum.

In certain embodiments, quantum dots of the sensor described herein are, for example, inorganic crystallites between 1 nm and about 1000 nm in diameter, preferably between about 2 nm and about 50 nm, more preferably about 5 nm to 20 nm, such as about 6, 7, 8, 9, 10, 11, 12, 13, 14, 15, 16, 17, 18, 19, or 20 nm. Such quantum dots include a “core” of one or more first semiconductor materials, and which may be surrounded by a “shell” of a second semiconductor material. A semiconductor nanocrystal core surrounded by a semiconductor shell is referred to as a “core/shell” semiconductor nanocrystal. The surrounded “shell” will most preferably have a bandgap greater than the bandgap of the core material and can be chosen so to have an atomic spacing close to that of the “core” substrate. The core and/or the shell material can be a semiconductor material including, but not limited to, those of the group II-VI (ZnS, ZnSe, ZnTe, CdS, CdSe, CdTe, HgS, HgSe, HgTe, MgTe and the like) and III-V (GaN, GaP, GaAs, GaSb, InN, InP, InAs, InSb, AlAs, AlP, AlSb, AlS, and the like) and IV (Ge, Si, Pb and the like) materials, and an alloy thereof, or a mixture thereof.

In certain aspects, a sensor comprises exactly one quantum dot. In certain embodiments, a sensor comprises more than one quantum dot, for example, 2, 3, 4, or 5 quantum dots. In certain embodiments, wherein the sensor comprises more than one quantum dot, the sensor comprises two or more types of quantum dots, each type having a distinct emission wavelength, e.g., independently selected from, for example, 490, 520, 545, 560, 580, 620, 655 nm. The availability of two distinct wavelength emissions (e.g., one or more quantum dots of wavelength 545 nm and one or more quantum dots with emission wavelength of 655 nm) may allow improvements in recording of changes in analyte concentration by using the ratio of the two distinct signals. Fluctuations in fluorescence that are common to both signals should theoretically cancel in a ratio. The detectable fluorescence emission of the quantum dot particles may fluctuate depending on variables including number of quantum dots, quantum dot location within the cell, photobleaching, and possible changes in excitation light intensity, all effects that can occur slowly and are not related to analyte presence or concentration. Therefore, effects including number of quantum dots, quantum dot location within the cell, photobleaching, and possible changes in excitation light intensity, may be attenuated.

In certain embodiments, the fluorescence signal of the quantum dot may trigger a detectable event within the cell. For example, fluorescence may in turn excite a secondary dye or quantum dot in the particle that easily generates reactive oxygen species (ROS). The ROS would then attack the cell, effectively stimulating necrosis (cell death), which may then be detected either visually or using markers sensitive to cell death. Alternatively, instead of including a secondary component within the particle, another particle may be added to the cell or cell culture. This additional particle may, for example, comprise a photo-degradable polymer membrane. When the fluorescent component fluoresces, the emitted light will rupture the secondary particle, releasing its contents. The contents may, for example, be a drug that is therapeutic or apoptotic, e.g., triggering another detectable event.

In certain aspects, the sensor of the application is a polymer film. In certain embodiments, the film comprises a polymeric matrix comprising a fluorescent component, a chromophore and moieties that chelate analytes. In certain embodiments, the film comprises multiple fluorescent components, chromophores and moieties that chelate analytes. In certain aspects, the film is a polymer matrix comprising one or more sensor particles of the invention. A sensor film may be deposited on any surface such as plastic, metal, paper or glass. The film may be deposited on an item such as a multi-well plate, a stirring rod, a Petri dish or sample cup. In certain embodiments, the film can be applied to a surface such as by painting or spraying the surface with the polymer film, or by immersing the surface in a solution or dispersion of the elements of the polymer film. In certain embodiments, the polymer film solidifies after the film has been applied to the surface. The polymer used in such films may be any one or more of the polymers described herein or any other suitable polymer. In certain embodiments, the film further comprises a biocompatible coating. The fluorescent component of the film may be one or more quantum dots.

The quantum dot of the sensor particle may be modified with a surface modifier, e.g., to alter one or more properties of the sensor particle, such as solubility, biocompatibility, or hydrophilicity/hydrophobicity. In certain embodiments, the surface modifier comprises one or more ligands that can bind reversibly with the quantum dot, while in other embodiments, the surface modification may be essentially irreversible. In certain embodiments, the surface modifier improves the lipophilicity of the quantum dot. In certain such embodiments, the ligand comprises an alkane such as decane-thiol.

In certain embodiments, the invention comprises methods of preparing particles selective for a chelatable analyte, comprising contacting a quantum dot with a polymeric precursor mixture including moieties that bind the chelatable analyte, and a chromophore. In certain embodiments, moieties are chosen which chelate glucose. In certain embodiments, the moieties that bind the chelatable analytes comprise boronic acids and/or boronic esters. In certain embodiments, the method further comprises coating the polymer matrix with a biocompatible layer.

Chemical Vapor Deposition (iCVD), a coating technology, may be used to deposit a layer that protects the sensors from the surrounding medium. The solventless nature of iCVD particle coating may offer an advantage over solution-based methods that rely on drying of a wet polymer solution. In certain embodiments, the iCVD particle coating employs a custom-designed rotating bed reactor that has been demonstrated to provide conformal coating of microspheres and nanoparticles without inducing aggregation. In certain embodiments, the primary monomer for the iCVD coatings of GSQDs is hydroxyethylmethacrylate (HEMA) monomer.

In certain embodiments, the iCVD coatings of the nanoparticles are pure polymer and no residual solvent is present, e.g., that may cause implant rejection, irritation, or other unwanted side effects. The coatings can be applied at room temperature in a single step, taking only a few minutes of total time. In certain embodiments, the composition can be controlled systematically by changing the gas feed mix and thickness can be controlled by in situ monitoring

In certain embodiments, the invention includes methods for detecting the presence of a chelatable analyte in a medium, comprising contacting a sensor particle of the invention with a medium, exposing the quantum dot to light energy that causes the quantum dot to emit photons and using a detector to detect the photons and determining the presence or absence of bound chelatable analyte based on the detected photons. In certain embodiments, the chelatable analyte is glucose. In certain embodiments, the light energy is selected from ultraviolet, infrared, near infrared or visible radiation. In certain embodiments, the light energy is ultraviolet. In certain embodiments, the medium comprises water, blood, plasma or urine. In certain embodiments, the method of detecting glucose with a sensor particle of the invention is performed in vitro.

In certain aspects, the invention provides a method for detecting an analyte in an animal using any of the sensor particles of the invention. In certain embodiments, the invention provides a method for detecting the presence of a chelatable analyte in an animal, comprising the steps of: contacting a sensor particle of the invention with an animal cell or tissue, wherein the sensor particle comprises at least one quantum dot and/or fluorescent dye; a polymer matrix comprising a polymer matrix including moieties that bind a chelatable analyte and a chromophore associated with the polymer matrix that binds to the moieties in the absence of the chelatable analyte; exposing the particles to light energy that causes the quantum dot and/or fluorescent dye to emit photons; using a detector to detect the photons; and determining the presence or absence of bound chelatable analyte based on the detected photons. In certain embodiments, the particle is implanted within the dermis or epidermis of an animal. In certain embodiments, the chelatable analyte is glucose.

In certain embodiments, the particle comprises a biocompatible layer. The term “particle” may refer to one or more sensor particle of the invention. In certain embodiments, the particle comprises many sensor particles. In certain embodiments, the particle comprises a fluorescent dye and/or a quantum dot. In certain embodiments, the particle comprises at least one quantum dot, a chromophore, and a polymer matrix. In certain embodiments, the photons emitted by the quantum dot in an excited state are absorbed by a chromophore in an unbound state but not absorbed by a chromophore in a bound state. In certain other embodiments, the photons emitted by the quantum dot in an excited state are absorbed by a chromophore in a bound state but not absorbed by a chromophore in an unbound state.

In certain embodiment, the method for detecting an analyte in an animal comprises implanting the particle below the surface of the epidermis or dermis of the animal. The particle may be implanted intracellularly, while in other embodiments, the sensors are implanted extracellularly. When implanted in tissues, the composition may be taken into a cell or remain external to a cell. The particle may be implanted between about 0.05 mm and about 4 mm below the surface of the epidermis or dermis of the animal. In certain embodiments, the particle is injected or surgically inserted within the dermis or epidermis of an animal. In certain embodiments, the particle is injected within the dermis or epidermis of the animal. In certain embodiments, the particle is injected in a solution. In certain embodiments, a particle solution comprises multiple particles. The particle solution may comprise particles with an average particle size between 10 nm and 10 microns. In certain embodiments, the particle solution comprises particles with an average particle size between 10 microns and 500 microns such as between 50 microns and 200 microns. In certain embodiments, the amount of signal decrease over time due to fouling and leaching for the implanted particle sensor is minimal.

In certain embodiments, the implanted particle produces an optical change upon contact with a chelatable analyte. In certain embodiments, the optical change is the appearance of a color upon chelation of the moieties of the particle with the chelatable analyte, For example, in certain embodiments, when a colorless particle comes into contact with the chelatable analyte glucose, the chelatable particle turns red. In certain embodiments, wherein the particle is implanted in the dermis or epidermis, the color change can be seen from the surface of the skin. In certain other embodiments, the sensor turns yellow, green, blue, purple or orange.

In certain embodiments, the particle emits photons when contacted by a chelatable analyte which can be detected spectrophotometrically. The particle may emit photons immediately upon making contact with the chelatable analyte. In certain embodiments, the particle may emit photons after a brief time such as 1-5 seconds upon making contact with the chelatable analyte. In an exemplary embodiment, when a particle comprising a quantum dot contacts glucose, the particle emits photons which can be detected with a spectrophotometer. In certain embodiments, the number of photons detected can be correlated with the amount of chelatable analyte present in a medium, e.g., blood. In certain embodiments, where the particle is implanted in the dermis or epidermis, the photons can be detected through the skin. In certain embodiments, the detector is a hand held unit that can be held near the skin to detect photons emitted from the sensor.

The epidermis may vary in thickness depending upon its location and the animal, but is generally up to about 1 mm thick in a human. When implanted in the epidermis, it is preferred that the particle is placed or implanted of from about 0.05 mm, about 0.06 mm, about 0.07 mm, about 0.08 mm, about 0.09 mm, about 0.10 mm, about 0.12 mm, about 0.14 mm, about 0.16 mm, about 0.18 mm, about 0.2 mm, about 0.22 mm, about 0.24 mm, about 0.26 mm, about 0.28 mm, about 0.30 mm, about 0.32 mm, about 0.34 mm, about 0.36 mm, about 0.38 mm, about 0.40 mm, about 0.42 mm, about 0.44 mm, about 0.46 mm, about 0.48 mm, about 0.50 mm, about 0.52 mm, about 0.54 mm, about 0.56 mm, about 0.58 mm, about 0.60 mm, about 0.62 mm, about 0.64 mm, about 0.66 mm, about 0.68 mm, about 0.70 mm, about 0.72 mm, about 0.74 mm, about 0.76 mm, about 0.78 mm, about 0.80 mm, about 0.82 mm, about 0.84 mm, about 0.86 mm, about 0.88 mm, about 0.90 mm, about 0.92 mm, about 0.94 mm, about 0.96 mm, or about 0.98 mm to about 1 mm below the outer surface of the epidermis of an animal. In another preferred aspect, the particle is implanted between about 0.1 mm and about 0.15 mm below the surface of the epidermis of the animal. Preferred animals include sheep, goats, cats, dogs, birds, cows, horses or pigs. A particularly preferred animal is a human.

When implanted in the epidermis of an animal, the particle may exist only days or weeks before the cells containing or surrounding the particle are shed from the animal. In certain embodiments, the particle would remain in the position in which it was implanted for 1-4 weeks. In certain embodiments, the particle will exist up to about 2 weeks before removal through natural replacement of epidermal layers.

In another embodiment, the particle is implanted in the dermis or dermal layers of an animal. The dermis may very in thickness depending upon its location and the animal, but is generally from about 1 mm to about 4 mm thick in a human. The dermis is located beneath the epidermis, often generally beginning about 1 mm beneath the epidermis, often generally beginning about 1 mm beneath the outer surface of the epidermis. The dermis does not actively shed, so that a particle may exist semi-permanently or permanently in an animal, i.e., remain in the dermis for months or years. Depending on the thickness of the epidermis and dermis, in certain embodiments, the particle may be implanted or placed in the dermis of from about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm, about 3.0 mm, about 3.1 mm, about 3.2 mm, about 3.3 mm, about 3.4 mm, about 3.5 mm, about 3.6 mm, about 3.7 mm, about 3.8 mm, about 3.9 mm, about 4.0 mm, about 4.1 mm, about 4.2 mm, about 4.3 mm, about 4.4 mm, about 4.5 mm, about 4.6 mm, about 4.7 mm, about 4.8 mm, or about 4.9 mm to about 5.0 mm beneath the outer surface of the epidermis. In certain preferred embodiments, the particle would be implanted of from about 1 mm to about 5 mm beneath the surface of the epidermis, with about 2 mm to about 3 mm being particularly preferred.

In certain embodiments, the particle sensor is coupled with an optical readout (e.g., placed over the implantation site). In certain embodiments, a small insulin pump is coupled to the optical readout device. The insulin pump may be configured such that the insulin pump is activated to deliver insulin if the optical readout detects a level of glucose above a predetermined value.

### EXAMPLES

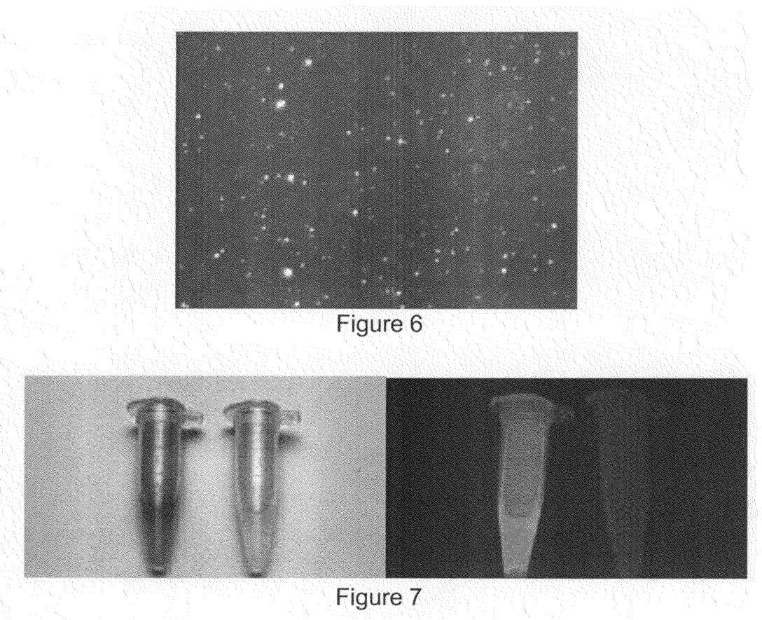

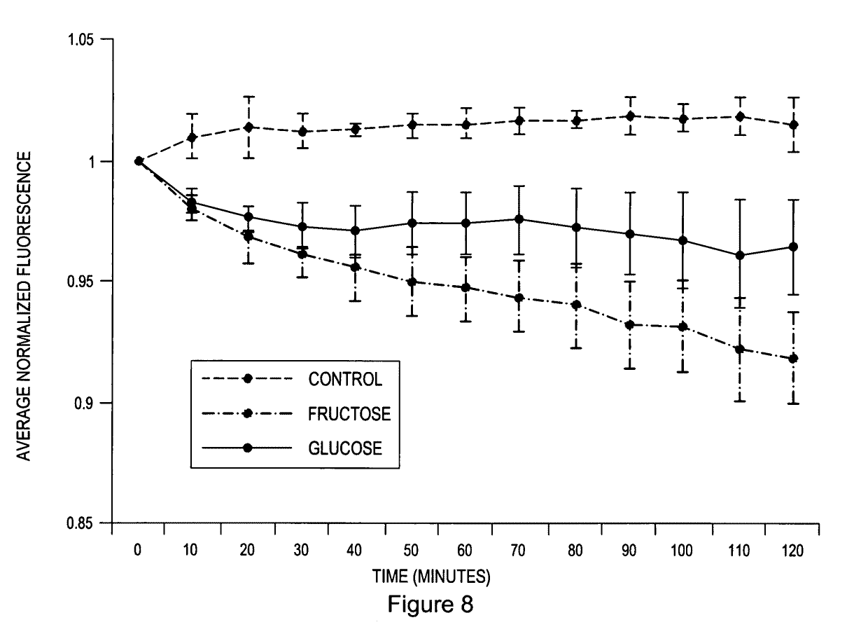

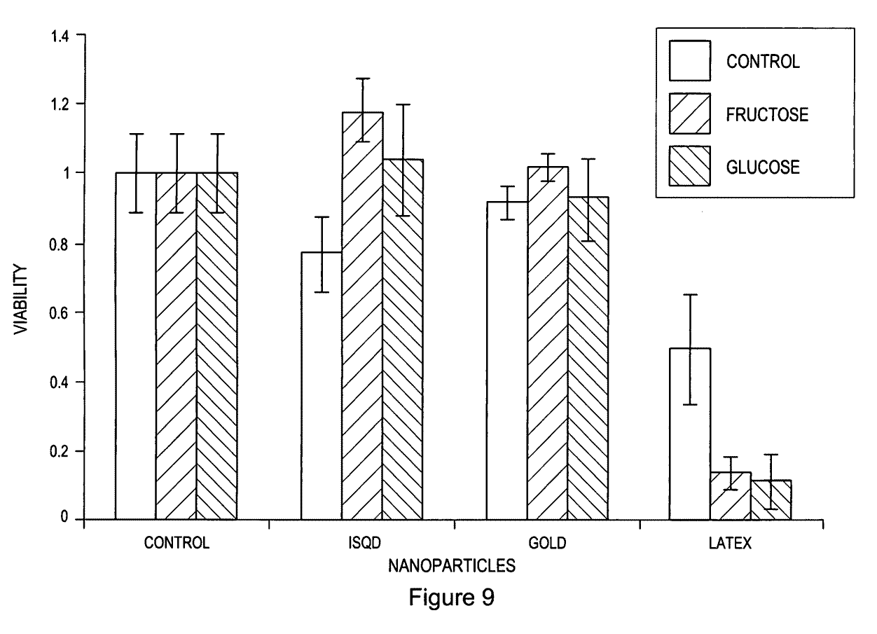

Nano-scale polymer-coated quantum dots: Commercially available quantum dots (Evident Technologies, Troy, N.Y.) were dispersed in a polymeric matrix. In order to make the dispersion homogeneous, a ligand exchange was performed to add a decane-thiol to the surface of the quantum dot. The alkylated surface proved more miscible with the lipophilic polymer matrix. After a homogeneous distribution was obtained, nanoscale sensors were produced by sonicating the polymeric matrix dissolved in THF, containing all of the sensing elements including quantum dots, in an aqueous solution of PEG-lipid surface modifier. The resulting nanosensor solution was filtered to remove larger pieces of polymer. The resulting sensor suspension fluoresced brightly when viewed in a wide-field fluorescence microscope (FIG. 6).
Inner-filter effect: Nanometer-sized glucose-sensitive quantum dots (GSQDs) in solution are shown in FIG. 7. The absorbance changes from purple to yellow are easily seen by eye in FIG. 7 (left). The same samples of nanosensors under UV excitation are shown in FIG. 7 (right). The sample that was visually purple does not absorb the 525 nm emission of the quantum dots and fluoresces brightly. The yellow GSQD absorbs the fluorescence emission of the quantum dot and has minimal emission.
Response to glucose: A polymer matrix containing the sensing components alizarin, pyrene boronic acid and additive, was immobilized to the bottom of a micro-well for calibration. Response to glucose and fructose was measured, the average±SEM is shown in FIG. 8, n=6 and 8 for control and monosaccharides, respectively.
Biocompatibility: In vitro biocompatibility studies produced no indications of cellular injury thus far. For instance, LIVE-DEAD assays showed no differences from controls in the amount of cell death. In addition, the degree of cytotoxicity was determined by incubating the nanosensors overnight with HEK 293 cells and measuring the degree of cellular injury with an MTT assay. These results were compared to other nanoparticles and are shown in FIG. 9. The ion-sensitive quantum dot (ISQD) nanosensors show no cellular toxicity compared to controls over the course of 72 hours after incubation. This result is also seen for 100 nm diameter gold nanoparticles,

### EQUIVALENTS

The present invention provides among other things sensor particles for detecting chelatable analytes and methods of use thereof. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

We claim:

1. A sensor particle for detecting the presence of a chelatable analyte, comprising:
a quantum dot;
a polymer matrix comprising a polymer including moieties that bind the chelatable analyte; and
a chromophore associated with the polymer matrix that binds to the moieties in the absence of the chelatable analyte.

2. The particle of claim 1, wherein the chelatable analyte is glucose.

3. The particle of claim 2, wherein the moieties that bind glucose comprise boronic acids and/or boronic esters.

4. The particle of claim 1, wherein the chromophore absorbs photons of a first wavelength when bound to the moieties, and absorbs photons of a second wavelength when released from the moieties.

5. The particle of claim 3, wherein the boronic acid and/or boronic ester moieties are covalently conjugated through linkers to the polymer matrix.

6. The particle of claim 1, wherein the moieties bind the chelatable analyte and the chromophore reversibly and competitively.

7. The particle of claim 1, wherein the chromophore is covalently conjugated to the polymer matrix.

8. The particle of claim 1, wherein photons emitted by the quantum dot in an excited state are absorbed by the chromophore in an unbound state but not by the chromophore in a bound state.

9. The particle of claim 1, further comprising a biocompatible coating disposed on at least a portion of the polymer matrix.

10. A method of preparing sensor particles selective for a chelatable analyte, comprising contacting a quantum dot with a polymeric precursor mixture including moieties that bind the chelatable analyte, and a chromophore.

11. The method of claim 10, wherein the chelatable analyte is glucose.

12. A sensor particle for detecting the presence of a chelatable analyte comprising:
a polymer matrix comprising a polymer including moieties that bind the chelatable analyte;
a chromophore associated with the polymer matrix that binds to the moieties in the absence of glucose.

13. The particle of claim 12, further comprising a fluorescent dye.

14. The particle of claim 12, wherein the bound chromophore emits photons at one wavelength and the unbound chromophore emits photons at a second wavelength.

15. The particle of claim 13, wherein photons emitted by the fluorescent dye in an excited state are absorbed by the chromophore in an unbound state but not by the chromophore in a bound state.

16. The particle of claim 13, wherein photons emitted by the fluorescent dye in an excited state are absorbed by the chromophore in a bound state but not by the chromophore in an unbound state.

17. The particle of any of claims 12-13, wherein the chelatable analyte is glucose.

18. The particle of claim 13, wherein the moieties that bind the chelatable analyte comprise boronic acids and/or boronic esters.

19. The particle of claim 13, wherein the moieties bind the chelatable analyte and the chromophore reversibly and competitively.

20. The particle of claim 13, further comprising a biocompatible coating disposed on at least a portion of the polymer matrix.

21. A method of preparing sensor particles selective for a chelatable analyte, comprising contacting a fluorescent dye with a polymeric precursor mixture comprising moieties that bind the chelatable analyte, and a chromophore.

22. The method of claim 21, wherein the chelatable analyte is glucose.

23. A method for detecting the presence of a chelatable analyte in a medium, comprising:
contacting a particle of claim 1 or 12 with the medium;
exposing the quantum dot to light energy that causes the quantum dot to emit photons;
using a detector to detect the photons; and
determining the presence or absence of bound chelatable analyte based on the detected photons.

24. A method for detecting the presence of a chelatable analyte in an animal, comprising the steps of:
contacting a sensor particle with an animal cell or tissue, wherein the sensor particle comprises at least one quantum dot and/or fluorescent dye; a polymer matrix comprising a polymer including moieties that bind a chelatable analyte and a chromophore associated with the polymer matrix that binds to the moieties in the absence of the chelatable analyte;
exposing the particles to light energy that causes the quantum dot and/or fluorescent dye to emit photons;
using a detector to detect the photons; and
determining the presence or absence of bound chelatable analyte based on the detected photons.

25. The method of claim 24, wherein the particle is implanted within the dermis or epidermis of the animal.

26. The method of claim 24, wherein the particle comprises at least one quantum dot.

27. The method of claim 24, wherein the particle comprises at least one fluorescent dye.

28. The method of claim 24, wherein the particle produces an optical change upon contact with a chelatable analyte.

29. The method of claim 24, wherein the moieties bind the chelatable analyte and the chromophore reversibly and competitively.

30. The method of claim 24 wherein the chelatable analyte is glucose.

31. The method of claim 24, wherein the chromophore absorbs photons of a first wavelength when bound to the moieties, and absorbs photons of a second wavelength when released from the moieties.

32. The method of claim 24, wherein photons emitted by the quantum dot in an excited state are absorbed by the chromophore in an unbound state but not by the chromophore in a bound state.

33. The method of claim 24, wherein photons emitted by the quantum dot in an excited state are absorbed by the chromophore in a bound state but not by the chromophore in an unbound state.

34. The method of claim 24, wherein the particle further comprises a biocompatible coating disposed on at least a portion of the polymer matrix.
